(19) United States
(12) Patent Application Publication
(10) Pub. No.: US 2023/0307115 A1
(43) Pub. Date: Sep. 28, 2023

(54) MACHINE LEARNING FOR EFFECTIVE PATIENT INTERVENTION

(71) Applicant: MatrixCare, Inc., Bloomington, MN (US)

(72) Inventors: Vivek KUMAR, Eden Prairie, MN (US); Coleen DANIELSON, Fergus Falls, MN (US)

(21) Appl. No.: 18/166,943

(22) Filed: Feb. 9, 2023

Related U.S. Application Data
(60) Provisional application No. 63/322,586, filed on Mar. 22, 2022.

(51) Int. Cl. G16H 20/70; G16H 50/30; G06F 40/40

(52) U.S. Cl. CPC G16H 20/70; G16H 50/30; G06F 40/40

(57) ABSTRACT

Techniques for improved machine learning are provided. User data describing a user is received, and a set of user attributes corresponding to a defined set of features is extracted from the user data. A risk score is generated by processing the set of user attributes using a trained machine learning model, where the risk score indicates a probability that the user has or will develop depression. One or more interventions are initiated for the user based on the risk score.

### CROSS-REFERENCE TO RELATED APPLICATIONS

This application claims priority to U.S. Provisional Patent Application No. 63/322,586, filed Mar. 22, 2022, the entire content of which is incorporated herein by reference in its entirety.

### INTRODUCTION

Embodiments of the present disclosure relate to machine learning. More specifically, embodiments of the present disclosure relate to using machine learning to evaluate and treat user depression.

In conventional healthcare settings, such as in residential care facilities (e.g., nursing homes), a wide variety of user, patient, or resident characteristics are assessed and monitored in an effort to reduce or prevent worsening any resident's condition. Of particular importance in many instances is identifying potential risks for depression among users, and driving targeted and rapid interventions when needed. However, depression is a tremendously complex concern, and has a myriad of causes and symptoms. Without appropriate identification and intervention, depression can lead to clinically significant negative outcomes and complications.

Conventionally, healthcare providers (e.g., doctors, nurses, caregivers, and the like) strive to identify potential depression signs using manual assessments (e.g., talking with the patient). However, such conventional approaches are entirely subjective (relying on the expertise of individual caregiver to recognize possible concerns), and frequently fail to identify those most in need of additional care. Further, given the vast complexity involved in clinical depression, it is simply impossible for healthcare providers to evaluate all relevant data in order to identify potential concerns.

Improved systems and techniques to automatically evaluate patients are needed.

### SUMMARY

According to one embodiment presented in this disclosure, a method is provided. The method includes: receiving user data describing a first user; extracting, from the user data, a first set of user attributes corresponding to a defined set of features, wherein at least depression first attribute of the first set of user attributes is generated by processing unstructured text using a first trained machine learning model; training a second machine learning model to generate risk scores based on the first set of user attributes, wherein the risk scores indicate probability that users have or will develop depression; and deploying the second trained machine learning model.

According to one embodiment presented in this disclosure, a method is provided. The method includes: receiving user data describing a first user; extracting, from the user data, a first set of user attributes corresponding to a defined set of features, wherein at least a first attribute of the first set of user attributes is generated by processing unstructured text using a first trained machine learning model; generating a first risk score by processing the first set of user attributes using a first trained machine learning model, wherein the first risk score indicates a probability that the first user has or will develop depression; and initiating one or more interventions for the first user based on the first risk score.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow for training machine learning models based on historical data.

FIG. 2 depicts an example workflow for generating risk scores and interventions using machine learning models.

FIG. 3 depicts an example workflow for preprocessing unstructured data for improved machine learning.

FIG. 4 is a flow diagram depicting an example method for generating training data for improved machine learning.

FIG. 5 is a flow diagram depicting an example method for training machine learning models to evaluate user depression.

FIG. 6 is a flow diagram depicting an example method for using trained machine learning models to use machine learning to generate risk scores and implement appropriate interventions.

FIG. 7 is a flow diagram depicting an example method for extracting user attributes for input to machine learning models.

FIG. 8 is a flow diagram depicting an example method for evaluating unstructured input data to improve machine learning.

FIG. 9 is a flow diagram depicting an example method for preprocessing unstructured input data to improve machine learning results.

FIG. 10 is a flow diagram depicting an example method for using machine learning to evaluate trends in user risk and implement appropriate interventions.

FIG. 11 is a flow diagram depicting an example method for using machine learning to evaluate and aggregate user risk.

FIG. 12 is a flow diagram depicting an example method for preprocessing unstructured input data to improve machine learning results.

FIG. 13 is a flow diagram depicting an example method for generating risk scores using trained machine learning models.

FIG. 14 is a flow diagram depicting an example method for training machine learning models to improve user risk evaluation.

FIG. 15 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved machine learning to detect and evaluate depression in users.

In some embodiments, a machine learning model (also referred to in some aspects as a risk model or a depression model) can be trained and used as an assessment tool for clinicians (e.g., nurses, caregivers, doctors, and the like) to assist in identifying users at risk of depression (e.g., patients, residents of a long-term care facility, and the like), thereby improving care, and preventing potentially significant negative outcomes. In some embodiments, by monitoring for changes in the conditions for each user, the system is able to identify those in need of additional care, and can assist with reallocating resources and driving targeted interventions to help mitigate, prevent, or reduce the effect of the risk or depression. In some embodiments, the machine learning model enables real-time insights, including alerts that notify the clinician or caretaker when there has been a change in condition with any user (e.g., within the past 24 hours) that affects potential depression, and provides the clinician with an actionable assessment and intervention details.

In conventional settings, caretakers must rely on subjective assessments (e.g., discussions with the users) to perform such analysis. In addition to this inherently subjective and inaccurate approach, some conventional systems rely on manual identification of potential risk factors in patient data, such as adverse diagnoses (e.g., terminal cancer). However, these risk factors are similarly subjective and uncertain. Moreover, the vast number and variety of factors that affect depression, as well as the significant amounts of data available for each user, render such analysis impossible to adequately perform manually or mentally. Aspects of the present disclosure can not only reduce or prevent this subjective review, but can further prevent wasted time and computational expense spent reviewing vast amounts of irrelevant data. Further, aspects of the present disclosure enable more accurate evaluations, more efficient use of computational resources, and overall improved outcomes for users who may be at risk.

Embodiments of the present disclosure can generally enable proactive and quality care for users, as well as dynamic and targeted interventions, and that help to prevent or reduce adverse events due to depression. This autonomous and continuous updating based on changing conditions with respect to individual users (as well as across groups of users, in some aspects) enables a wide variety of improved results, including not only improved outcomes for the users (e.g., reduced depression, early identification of potential issues, targeted interventions, and the like) but also improved computational efficiency and accuracy of the evaluation and solution process.

In some embodiments, a variety of historical user data can be collected and evaluated to train one or more machine learning models. During such training, the machine learning model(s) can learn a set of features (e.g., user attributes) and/or a set of weights for such features. These features and weights can then be used to automatically and efficiently process new user data in order to identify potential concerns for depression. In some aspects, the model may be trained during a training phase, and then be deployed as a static model that, once deployed, remains fixed. In other embodiments, the model may be refined or updated (either periodically or upon specified criteria). That is, during use in detecting and intervening in potential depressions, the model may be refined based on feedback from users, caregivers, and the like. For example, if a clinician indicates that a user has depression (regardless of whether the model predicted the diagnosis), the model may be refined based on this indication. Similarly, if a clinician indicates that the user does not have depression, the model may be refined to reflect this new data.

In embodiments, the machine learning model(s) can generally be used to generate risk or depression scores or classifications for a given user based on their attributes. For example, the system may determine or extract the user's demographics (e.g., age, gender, marital status, and the like), diagnoses, clinical assessments, medications, and the like. In some embodiments, the attributes include both structured data (such as defined diagnoses) as well as unstructured data (e.g., natural language notes written by the user or a clinician). These attributes can then be processed as input to the machine learning model(s), which can generate a risk score (also referred to as a depression score or a depression risk score in some aspects) and/or classification (e.g., indicating low, moderate, or high risk of depression) for the user. In some embodiments, the models can additionally be used to monitor and identify changes or trends in the risk (e.g., over time), as well as identifying the specific attribute(s) that are causing a heightened risk. In these ways, the machine learning models can help drive specific and targeted interventions and assistance to improve user outcomes and reduce wasted resources and computational expense.

### Example Workflow for Training Machine Learning Models Using Historical Data

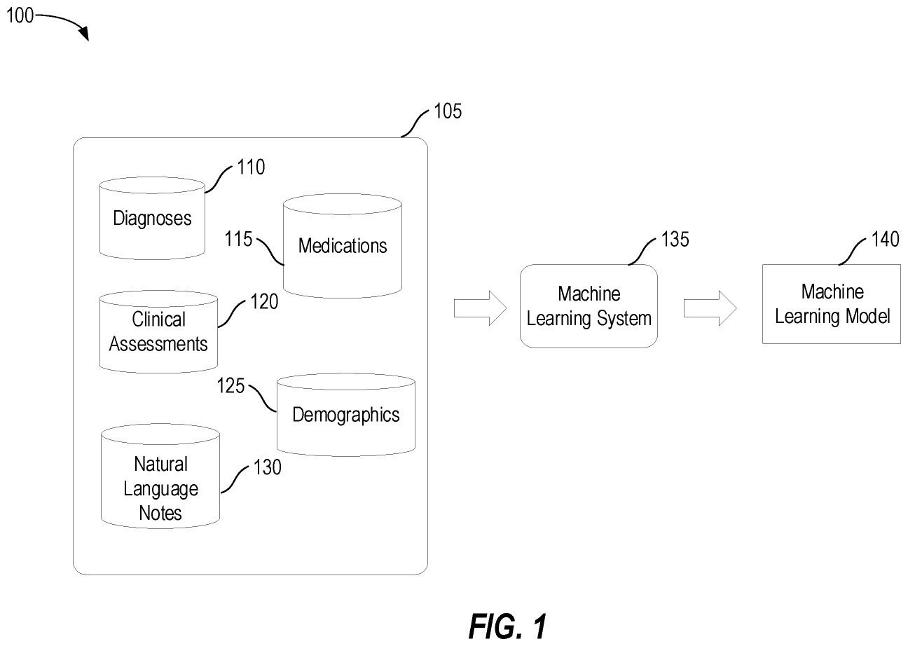

FIG. 1 depicts an example workflow 100 for training machine learning models based on historical data.

In the illustrated workflow 100, a set of historical data 105 is evaluated by a machine learning system 135 to generate one or more machine learning models 140. In embodiments, the machine learning system 135 may be implemented using hardware, software, or a combination of hardware and software. The historical data 105 generally includes data or information associated with one or more users (also referred to as patients or residents) from one or more prior points in time. That is, the historical data 105 may include, for one or more users, a set of one or more snapshots of the resident's characteristics or attributes at one or more points in time. For example, the historical data 105 may include attributes for a set of users residing in one or more long-term care facilities. In some embodiments, the historical data 105 includes indications of when any of the user attributes changed (e.g., records indicating the updated attributes whenever a change occurs). The historical data 105 may generally be stored in any suitable location. For example, the historical data 105 may be stored within the machine learning system 135, or may be stored in one or more remote repositories, such as in a cloud storage system.

In the illustrated example, the historical data 105 includes, for each user reflected in the data, a set of one or more diagnoses 110, medications 115, clinical assessments 120, demographics 125, and natural language note 130. In some embodiments, as discussed above, the historical data 105 includes data at multiple points in time for each user. That is, for a given user, the historical data 105 may include multiple sets of diagnoses 110 (one set for each relevant point in time), and the like. In some embodiments, the data contained within the diagnoses 110, medications 115, clinical assessments 120, demographics 125, and natural language notes 130 are associated with timestamps or other indications of the relevant time or period for the data. In this way, the machine learning system 135 can identify the relevant data for any given point or window of time. For example, if the diagnoses 110 indicate depression at a given time, the machine learning system 135 can identify all the relevant data surrounding this time (e.g., at the time, within a predefined window before the time, such as one month prior, and the like).

In some embodiments, the historical data 105 may be collectively stored in a single data structure. For example, the diagnoses 110, medications 115, clinical assessments 120, demographics 125, and natural language notes 130 may each be represented in a user profile (with indications of any changes over time), or as a sequence of structures (e.g., a set of profiles or forms, each corresponding to a particular point or window in time and containing attributes for that time). In some portions of the present discussion, the various components of the historical data 105 are described with reference to a single user for conceptual clarity (e.g., diagnoses 110 of a single user). However, it is to be understood that the historical data 105 can generally include such data for any number of users.

As discussed in more detail below, the diagnoses 110 generally correspond to a set of one or more specified diagnoses or disorders. In such an embodiment, the historical data 105 can indicate, for each specified disorder or diagnosis, whether the corresponding user has been diagnosed with the specified disorder (at the relevant time). In some embodiments, the diagnoses 110 are curated or selected based on their impact on potential depression. For example, in one aspect, a user (e.g., a clinician) may manually specify disorders that have a high impact on future depression. In some embodiments, some or all of the diagnoses may be inferred or learned (e.g., using one or more feature selection techniques). For example, one or more machine learning models or feature selection algorithms may be used to identify specific disorders (or to determine dynamic weights for each disorder) based on their impact on the risk of depression.

As discussed in more detail below, the medications 115 generally correspond to a set of one or more specified therapies (e.g., medications or other interventions). The medications 115 can generally indicate, for each specified medication, therapy, or intervention, whether the corresponding user receives (or should receive) the medication, therapy, or intervention. In some embodiments, the medications 115 are curated or selected based on their impact on depression. For example, in one aspect, a user (e.g., a clinician) may manually specify medications that are correlated with or have a high impact on risk for depression. In some embodiments, some or all of the medications or therapies may be inferred or learned (e.g., using one or more feature selection techniques), as discussed above.

As discussed in more detail below, the clinical assessments 120 generally correspond to a set of one or more specified assessments (e.g., prepared by a caregiver or clinician) of conditions relating to the functional state of the user. The clinical assessments 120 can generally indicate, for each specified condition, whether the corresponding resident has been assessed with the specified condition. For example, the clinical assessments 120 may indicate whether the resident has been assessed with weight gain or loss, sleeping disturbances or changes, food intake increase or decrease, pain, isolation, behavioral issues, and the like. In some embodiments, the clinical assessments 120 are curated or selected based on their impact on depression risk. For example, in one aspect, a user (e.g., a clinician) may manually specify assessments or conditions that have a high impact on depression (or are highly correlated with depression). In some embodiments, some or all of the conditions or assessments may be inferred or learned (e.g., using one or more feature selection techniques), as discussed above.

As discussed in more detail below, the demographics 125 of the user can generally indicate demographic characteristics of the user, such as their age, gender, marital status (e.g., single, married, widowed, recently-widowed, and the like), race, and the like. In some embodiments, the demographics 125 can further indicate information such as familial relationships (e.g., whether the user has close family or friends, whether they receive visitors, and the like). In some embodiments, the specific demographics 125 are curated or selected based on their impact on depression risk. For example, in one aspect, a user (e.g., a clinician) may manually specify demographic characteristics that are correlated with depression risk. In some embodiments, some or all of the characteristics may be inferred or learned (e.g., using one or more feature selection techniques), as discussed above.

As discussed in more detail below, the natural language notes 130 can generally include natural language text written by one or more users (e.g., by a user about themselves, or by a clinician about the user). For example, during or after a caregiving visit, a clinician may write a freehand note indicating their general impression of the user's state, any complains given, and the like. In some embodiments, as discussed in more detail below, these natural language notes 130 can be evaluated (e.g., using natural language processing techniques or machine learning models) to help quantify the potential risk of depression for the user.

Although the illustrated historical data 105 includes several specific components including diagnoses 110, medications 115, clinical assessments 120, demographics 125, and natural language notes 130, in some embodiments, the historical data 105 used by the machine learning system 135 may include fewer components (e.g., a subset of the illustrated examples) or additional components not depicted. Additionally, though the illustrated example provides general groupings of attributes to aid understanding, in some embodiments, the historical data 105 may be represented using any number of groups. That is, the individual attributes (e.g., individual diagnoses) may simply be used as input to the machine learning system 135, without reference to any larger grouping or component.

Additionally, though the above discussion relates to receiving specified sets of data (e.g., specified diagnoses), in some aspects, the machine learning system 135 may receive a broader set of data (e.g., all diagnoses of the residents). The machine learning system 130 may then select which subset of the data to consider (e.g., using feature selection techniques, as discussed above, or based on the defined set of features to be used), or may assign weights to each feature (e.g., using machine learning) to generate accurate depression risk scores.

As illustrated, the machine learning system 135 generates one or more machine learning models 140 based on the historical data 105. The machine learning model 140 generally specifies a set of weights for the various features or attributes of the historical data 105. In some embodiments, the machine learning model 140 specifies weights specifically for each individual feature (e.g., for each diagnosis in the set of diagnoses 110). For example, a first diagnosis may be associated with a lower weight than a second diagnosis. Similarly, in some embodiments, the machine learning model 140 specifies different weights depending on the severity of the feature (e.g., depending on the severity of the disorder or diagnosis). In some embodiments, the machine learning model 140 specifies weights for groups of features (e.g., a first weight for the diagnoses 110, a second weight for the medications 115, and so on).

In at least one embodiment, the machine learning model 140 can specify weights for one or more individual features, as well as weights for one or more broader categories. For example, the various diagnoses 110 may be individually weighted to generate an overall score or value for the diagnoses 110 input (e.g., a weighted sum or average). This value for the diagnoses 110 input can then be weighted (along with the other groups of input data) to generate an overall risk of depression for the user (e.g., using a weighted sum or average).

In some embodiments, the specific features considered by the machine learning model 140 (e.g., the specific diagnoses) are manually defined and curated. For example, the specific features may be defined by a subject-matter expert. In other embodiments, the specific features are learned during a training phase.

For example, the machine learning system 135 may process the historical data 105 for a given user at a given time as input to the machine learning model 140, and compare the generated depression risk to the ground-truth (e.g., an indication as to whether the user was actually diagnosed with depression). The difference between the generated and actual depression risk can be used to refine the weights of the machine learning model 140, and the model can be iteratively refined (e.g., using data from multiple users and/or multiple points in time) to generate accurate risk scores.

In some embodiments, during or after training, the machine learning system 135 may prune the machine learning model 140 based in part on the learned weights. For example, if the learned weight for a given feature (e.g., a specific demographic) is below some threshold (e.g., within a threshold distance from zero), the machine learning system 135 may determine that the feature has no impact (or negligible impact) on the risk of depression. Based on this determination, the machine learning system 135 may cull or remove this feature from the machine learning model 140 (e.g., by removing one or more neurons, in the case of a neural network). For future evaluations, the machine learning system 135 need not receive data relating to these removed features (and may refrain from processing or evaluating the data if it is received). In this way, the machine learning model 140 can be used more efficiently (e.g., with reduced computational expense and latency) to yield accurate risk evaluations.

In some embodiments, the machine learning system 135 can generate multiple machine learning models 140. For example, a separate machine learning model 140 may be generated for each facility (e.g., with a unique model for each specific long-term residential care facility), or for each region (e.g., with a unique model for each country). This may allow the machine learning system 135 to account for facility-specific, region-specific, or culture-specific changes (e.g., due to climate, average sunlight, and the like). In other embodiments, the machine learning system 135 generates a universal machine learning model 140. In at least one embodiment, the machine learning model 140 may use similar considerations (e.g., location, region, and the like) as an input feature.

In some embodiments, the machine learning system 135 outputs the machine learning model 140 to one or more other systems for use. That is, the machine learning system 135 may distribute the machine learning model 140 to one or more downstream systems, each responsible for one or more facilities. For example, the machine learning system 135 may deploy the machine learning model 140 to one or more servers associated with specific care facilities, and these servers may use the model to evaluate depression risks for residents at the specific facility. In at least one embodiment, the machine learning system 135 can itself use the machine learning model to evaluate user depression risks across one or more locations.

### Example Workflow for Generating Risk Scores and Interventions Using Machine Learning Models

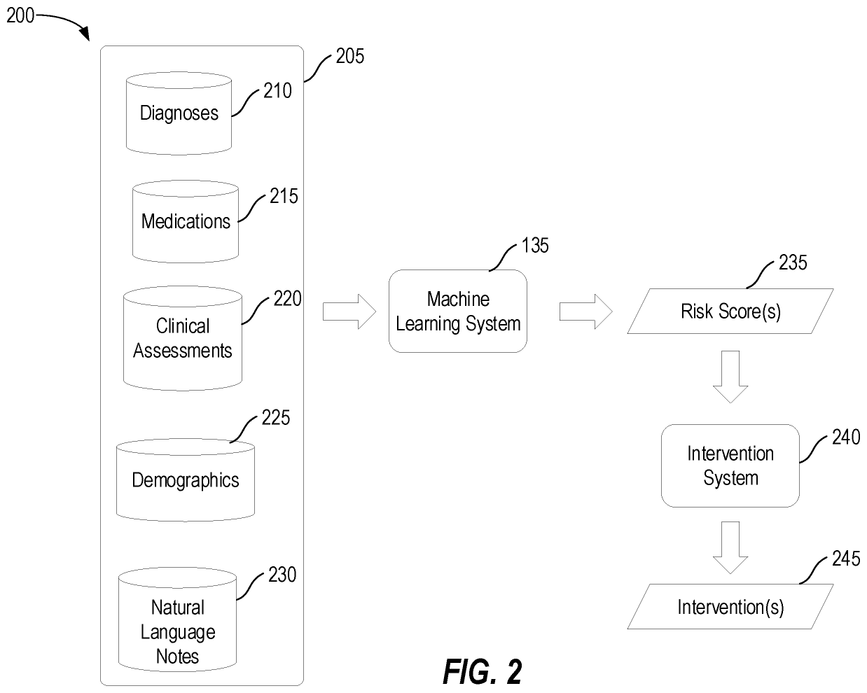

FIG. 2 depicts an example workflow 200 for generating risk scores and interventions using machine learning models.

In the illustrated workflow 200, a set of user data 205 is evaluated by a machine learning system 135 using one or more machine learning models (e.g., machine learning model 140 of FIG. 1) to generate one or more risk score(s) 235. In embodiments, the machine learning system 135 may be implemented using hardware, software, or a combination of hardware and software. In some embodiments, the machine learning system 135 that uses the machine learning model(s) is the same as the system that trains the machine learning model. In other aspects, as discussed above, the machine learning system 135 may differ from the system that trained the model.

The user data 205 generally includes data or information associated with one or more users (also referred to as patients or residents). That is, the user data 205 may include, for one or more users, a set of one or more snapshots of the user's characteristics or attributes at one or more points in time. For example, the user data 205 may include attributes for a set of residents residing in one or more long-term care facilities. The user data 205 may generally be stored in any suitable location. For example, the user data 205 may be stored within the machine learning system 135, or may be stored in one or more remote repositories, such as in a cloud storage system. In at least one embodiment, the user data 205 is distributed across multiple data stores.

In the illustrated example, the user data 205 includes, for each user reflected in the data, a set of one or more diagnoses 210, medications 215, clinical assessments 220, demographics 225, and natural language notes 230. In some embodiments, the data contained within the diagnoses 210, medications 215, clinical assessments 220, demographics 225, and natural language notes 230 are associated with timestamps or other indications of the relevant time or period for the data. In this way, the machine learning system 135 can generate a corresponding risk score 235 based on the relevant attributes at a given time.

As discussed above and in more detail below, the diagnoses 210 generally include information relating to a set of one or more diagnoses or disorders with respect to one or more users, the medications 215 generally include information relating to medications that the user(s) receive or have been prescribed, the clinical assessments 220 generally include information relating to whether one or more specified conditions (e.g., noted by a caregiver or clinician) have been noted for the user(s), the demographics 225 generally include information relating to characteristics or demographics of the user(s) (e.g., age, marital status, and the like), and the natural language note 230 generally include natural language text written by the user(s) and/or or written about the user(s) (e.g., by a clinician).

Although the illustrated user data 205 includes several discrete components for conceptual clarity, in some embodiments, the user data 205 used by the machine learning system 135 may include fewer components (e.g., a subset of the illustrated examples) or additional components not depicted. Additionally, though the illustrated example provides general groupings of attributes to aid understanding (e.g., grouping attributes as diagnoses, medications, and the like), in some embodiments, the user data 205 may be represented using any number of groups. That is, the individual attributes (e.g., individual diagnoses) may simply be used as input to the machine learning system 135, without reference to any larger grouping or component.

Additionally, though the above discussion relates to receiving specified sets of data (e.g., specified diagnoses), in some aspects, the machine learning system 135 may receive a broader set of data (e.g., all diagnoses of the users) and select which subset of the data to consider (e.g., based on the features specified in the machine learning model).

In the illustrated example, the user data 205 is used to generate one or more risk scores 235. The risk scores 235 can generally include one or more scores for one or more users. For example, in one aspect, the risk scores 235 can include one score for each respective resident of a plurality of residents (e.g., in a single facility). In one aspect, the risk scores 235 can include multiple scores for a single user (or for each user), where each score is associated with a corresponding point or window in time (with a corresponding set of attributes from the user data 205). As discussed above, the risk scores 235 can generally indicate the risk of depression for the user (e.g., the probability that the user currently has, or will develop, depression in the near future, such as within one month).

As discussed above, the machine learning system 135 can generate the risk scores 235 by identifying or extracting the relevant user attributes from the user data 205 (e.g., the relevant diagnoses, as indicated by the machine learning model), and processing these attributes using the weights and architecture of the machine learning model to generate an overall risk score 235 for the user based on the attributes. In some embodiments, the risk score 235 can additionally or alternatively include a classification or category (e.g., low, moderate, or high), determined based on one or more threshold values for the risk score.

In some embodiments, the machine learning system 135 can generate a new risk score 235 for each user periodically (e.g., daily). In some embodiments, the machine learning system 135 generates a new risk score 235 whenever new data becomes available (or when the user data 205 changes). For example, when a new resident enters a residential care facility, the machine learning system 135 may use their current attributes to generate an initial risk score. As another example, whenever a resident's attributes change (e.g., due to a newly-received diagnosis and/or a removed diagnosis), the machine learning system 135 may automatically detect the change and generate an updated risk score 235.

In embodiments, the risk scores 235 can be used for a wide variety of applications. In some embodiments, the risk scores 235 are used to define or predict resource allocations, interventions, and the like. In the illustrated example, the risk scores 235 are optionally provided to an intervention system 240, which can generate one or more interventions 245 based on the risk scores 235. In some embodiments, the intervention system 240 can identify changes in the risk score 235 of each user. In one such embodiment, one or more of the generated interventions 245 may include alerts when the risk score of a given user changes (e.g., when it increases). For example, when new data is received for a given user, and this new data causes the user's depression risk score 235 to increase (or increase above some threshold value or percentage of change), the intervention system 240 may transmit an alert (e.g., to one or more clinicians). In some embodiments, the alert may include information relating to the change, such as what caused the score to increase, when the increased occurred, the magnitude of the increase, and the like.

In at least one embodiment, the alert may include instructions or suggestions with respect to specific prophylactic interventions 245 for the user, such as increased monitoring or check-ins by caregivers, modified meal content and/or schedule, renewed or more frequent clinical assessments for changing conditions, therapy sessions, other interventions selected based on the underlying risk factors of the user, and the like.

Advantageously, the automatically generated risk scores 235 and/or interventions 245 can significantly improve the outcomes of the users, helping to identify depression risks at early stages, thereby preventing further deterioration and significantly reducing harm. Additionally, the autonomous nature of the machine learning system 135 enables improved computational efficiency and accuracy, as the risk scores 235 and/or interventions 245 are generated objectively (as opposed to the subjective judgment of clinicians or other users), as well as quickly and with minimal computational expense. That is, as the scores can be automatically updated whenever new data is available, users need not manually retrieve and review the relevant data (which incurs wasted computational expense, as well as wasted time for the user).

Further, in some embodiments, the machine learning system 135 can regenerate risk scores 235 during specified times (e.g., off-peak hours, such as overnight) to provide improved load balancing on the underlying computational systems. For example, rather than requiring caregivers to retrieve and review resident data for a facility each morning to determine if anything significant occurred overnight or the previous day, the machine learning system 135 can automatically identify such changes, and use the machine learning model(s) to regenerate risk scores 235 before the shift begins. This can transfer the computational burden, which may include both processing power of the storage repositories and access terminals, as well as bandwidth over one or more networks, to off-peak times, thereby reducing congestion on the system during ordinary (e.g., daytime) use and taking advantage of extra resources that are available during the non-peak (e.g., overnight) hours.

In these ways, embodiments of the present disclosure can significantly improve user outcomes while simultaneously improving the operations of the computers and/or networks themselves (at least through improved and more accurate scores, as well as better load balancing of the computational burdens).

### Example Workflow for Preprocessing Unstructured Data for Improved Machine Learning

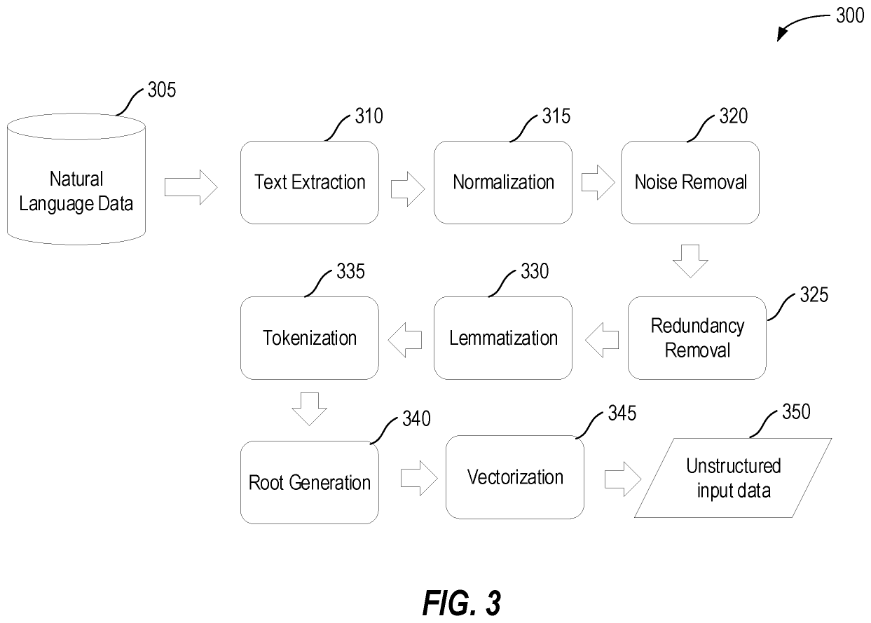

FIG. 3 depicts an example workflow 300 for preprocessing unstructured data for improved machine learning. In some embodiments, the workflow 300 may be performed to process natural language data 305 for input to one or more machine learning models. In some embodiments, the workflow 300 is performed by one or more remote systems (e.g., by a cloud-based service). In other embodiments, the workflow 300 is performed by a machine learning system, such as machine learning system 135 of FIGS. 1 and 2. The workflow 300 may be used during training of machine learning models (e.g., to generate the training input) and/or during inferencing using the models (e.g., as input to generate a risk score for a user). That is, the workflow 300 may be used to transform or preprocess any natural language input, prior to it being used as input to the machine learning model(s) during training or inferencing.

In the illustrated workflow 300, natural language data 305 is received for processing to generate unstructured input data 350. In some embodiments, the workflow 300 is referred to as preprocessing to indicate that it is used to transform, refine, manage, or otherwise modify the natural language data 305 to improve its suitability for use with machine learning systems (or other downstream processing). In some embodiments, the natural language data 305 corresponds to clinical notes, such as natural language notes 130 of FIG. 1 and/or natural language notes 230 of FIG. 2.

In some embodiments, preprocessing the data in the natural language notes 305 may improve the ML training process by making the data more compatible with natural language processing, and ultimately for consumption by the ML model during training. Preprocessing can generally include a variety operations. Though the illustrated workflow 300 depicts a series of operations being performed sequentially for conceptual understanding, in embodiments, some or all of the operations may be performed in parallel. Similarly, in embodiments, the workflow 300 may include additional operations not depicted, or may include a subset of the depicted operations.

In the illustrated example, the natural language data 305 can first undergo text extraction 310. The text extraction 310 generally corresponds to extracting natural language text from an unstructured portion of the natural language data 305. For example, if the natural language data 305 includes a set of clinical notes (e.g., notes written by a clinician describing an encounter with a user or patient), the text extraction 310 can include identifying and extracting these notes for evaluation. In some aspects, the notes may further include structured or semi-structured data that can undergo more traditional processing as needed, such as a timestamp indicating when the note was written or revised, an indication of the specific user about whom the note was written, the author of the note, and the like.

The normalization 315 can generally a wide variety of text normalization processes, such as converting all characters in the extracted text to lowercase, converting accented or foreign language characters to ASCII characters, expanding contractions, converting words to numeric form where applicable, converting dates to a standard date format, and the like.

Noise removal 320 can generally include identification and removal of portions of the extracted text that do not carry meaningful or probative value. That is, noise removal 320 may include removing characters, portions, or elements of the text that are not useful or meaningful in the ultimate computing task (e.g., computing a risk score), and/or that are not useful to human readers. For example, the noise removal 320 may include removing extra white or blank spaces, tabs, or lines, removing tags such as HTML tags, and the like.

Redundancy removal 325 may generally correspond to identifying and eliminating or removing text corresponding to redundant elements (e.g., duplicate words), and/or the reduction of a sentence or phrase to a portion thereof that is most suitable for machine learning training or application. For example, the redundancy removal 325 may include eliminating verbs (which may be unhelpful in the machine learning task), conjunctions, or other extraneous words that do not aid the machine learning task.

Lemmatization 330 can generally include stemming and/or lemmatization of one or more words in the extracted text. This may include converting words from their inflectional or other form to a base form. For example, lemmatization 330 may include replacing “holding,” “holds,” and “held” with the base form “hold.”

In one embodiment, tokenization 335 includes transforming or splitting elements in the extracted text (e.g., strings of characters) into smaller elements, also referred to as “tokens.” For example, the tokenization 335 may include tokenizing a paragraph into a set of sentences, tokenizing a sentence into a set of words, transforming a word into a set of characters, and the like. In some embodiments, tokenization 335 can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

In an embodiment, root generation 340 can include reducing portion of the extracted text (e.g., a phrase or sentence) to its most relevant n-gram (e.g., a bigram) or root for downstream machine learning training and/or application.

Vectorization 345 may generally include converting the text into one or more objects that can be represented numerically (e.g., into a vector or tensor form). For example, the vectorization 345 may use one-hot encodings (e.g., where each element in the vector indicates the presence or absence of a given word, phrase, sentiment, or other concept, based on the value of the element). In some embodiments, the vectorization 345 can correspond to any word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate depression risk scores).

As illustrated, the various preprocessing operations in the workflow 300 result in generation of unstructured input data 350. That is, the unstructured input data 350 corresponds to unstructured natural language data 305 that has undergone various preprocessing to improve its use with downstream machine learning models. The preprocessing workflow 300 can generally include any other suitable techniques for making text ingestion more efficient or accurate (either in a training phase of a machine learning model, or while generating an inference or prediction using a trained model). Generally, improving the results of this natural language processing can have significant positive impacts on the computational efficiency of processing the data downstream, as well as the eventual accuracy of the trained machine learning model(s).

In some embodiments, as discussed below, this unstructured input data 350 can be processed using one or more trained machine learning models to generate a score, for each note in the natural language data 305, indicating a likelihood, probability, or degree with which the note indicates that the user may be depressed.

### Example Method for Generating Training Data for Improved Machine Learning

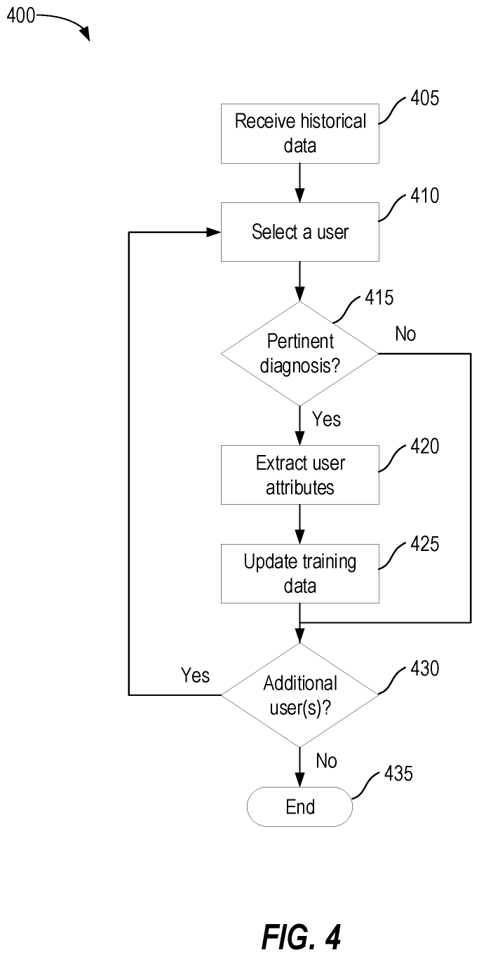

FIG. 4 is a flow diagram depicting an example method 400 for generating training data for improved machine learning. In the illustrated embodiment, the method 400 is performed by a machine learning system, such as the machine learning system 135 of FIG. 1. In other embodiments, the method 400 can be performed by other systems, such as training systems, data generation and/or aggregation systems, and the like.

At block 405, the machine learning system receives historical data (e.g., historical data 105 of FIG. 1). In an embodiment, the received historical data can generally include data or information associated with one or more users (also referred to as patients or residents) from one or more prior points in time. That is, the historical data may include, for one or more users, a set of user characteristics or attributes at one or more points in time. In some embodiments, the historical data includes attributes for a set of residents residing in one or more long-term care facilities.

In some embodiments, the received historical data includes user data corresponding to a defined set of features to be used to generate machine learning model to evaluate depression risks. These features may include, for example, specified diagnoses, specified clinical assessments and/or conditions, specified medications, specified demographics, features relating to natural language notes, and the like. In some embodiments, the historical data can include additional data beyond these features (e.g., information about all medications that one or more users have been prescribed, regardless of the specific medications used in the machine learning model). In one such embodiment, the machine learning system can identify and extract the relevant attributes or data based on the indicated features used for the machine learning model. In other embodiments, the received historical data may include only the specific data corresponding to the indicated features (e.g., another system may filter the historical data based on the features, thereby protecting data that is not needed to build the model).

In some embodiments, the historical data can include one or more indications, for each user reflected in the data, as to whether the user had or has depression. For example, these indications may be contained within the diagnoses portion of the historical data 405.

At block 410, the machine learning system selects one of the users reflected in the historical data. Generally, this selection can be performed using any suitable technique (including randomly or pseudo-randomly), as all of the users will be evaluated during the method 400. Although the illustrated example depicts an iterative or sequential selection and evaluation of each user, in some aspects, some or all of the method 400 may be performed in parallel. For example, rather than selecting and evaluating the data for each user individually, the machine learning system may first identify all users that satisfy the criteria (discussed below), then process the data from each (sequentially or in parallel).

At block 415, the machine learning system determines whether the selected user has one or more pertinent diagnoses. These pertinent diagnoses may generally correspond to a set of predefined depression-related diagnoses. In one such embodiment, the machine learning system may search electronic health records (EHR) of the user (reflected in the historical data) for a set of International Classification of Disease (ICD) codes, such as ICD-10 or ICD-11 codes. For example, the machine learning system may determine whether the user's data includes ICD-10 codes in the F32 or F33 ranges. Generally, such codes are indicative that a clinician has diagnosed the user with one or more depressive disorders.

If, at block 415, the machine learning system determines that the user has never received one or more of the pertinent diagnoses, the method 400 continues to block 430. That is, the machine learning system may discard the user's data (e.g., refrain from further processing of it), as the user has not suffered from depression. Although the illustrated example depicts discarding this data (thereby training the model based only on data from users who have suffered depression at some point), in some aspects, the machine learning system may instead use all of the historical data to train the model(s). That is, the machine learning system may use the data for users who have not had depression to refine the model (e.g., as a negative exemplar of the type of attributes that are not correlated with depression), while data from users who have had depression are also used to refine the model (e.g., as positive exemplars of the type of attributes that are correlated with depressive disorders).

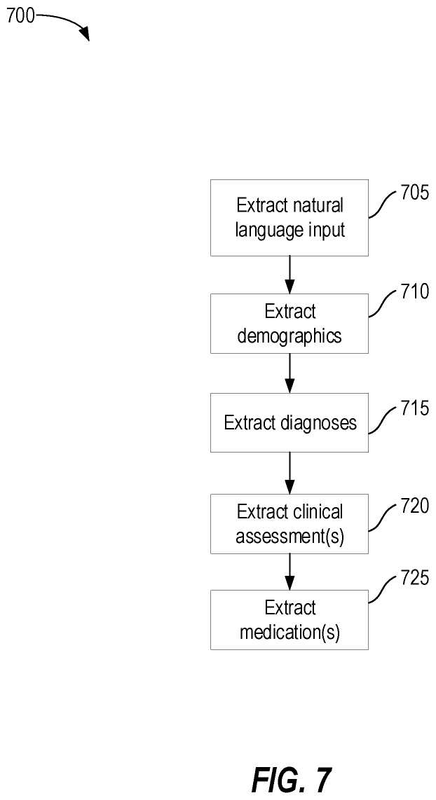

Returning to block 415, if the machine learning system determines that the user's data indicates at least one pertinent diagnosis, the method 400 continues to block 420. At block 420, the machine learning system extracts a set of user attributes, for the selected user, based on the pertinent diagnosis (or diagnoses). For example, for each pertinent diagnosis detected in the historical data of the selected user (or for each time when one or more pertinent diagnoses were entered into the user data), the machine learning system may extract relevant attributes corresponding to a defined window of time prior to the diagnosis (e.g., in the month leading up to the diagnosis). This can allow the machine learning system to generate a training data set that indicates sets of attributes that immediately-preceded a diagnosis of one or more depressive disorders. As discussed above and in more detail below with reference to FIG. 7, the attribute extraction may generally include extracting data relating to natural language notes, demographics, diagnoses, clinical assessments, medications, and the like.

At block 425, the machine learning system updates the training data to indicate these extracted attributes (and, in some aspects, information relating to the eventual diagnosis, such as the timing of the diagnosis with respect to the attribute(s)). In some embodiments, if multiple pertinent diagnoses are identified for a given user at different times in the historical data, the machine learning system can extract multiple sets of attributes, one for each point or window in time when a pertinent diagnosis was made.

In this way, the machine learning system can build a training data set that includes relevant attributes which immediately preceded a diagnosis of one or more depressive disorders. This data can be used to train one or more machine learning models, as discussed in more detail below, to predict depression risks for users.

At block 430, the machine learning system determines whether data for at least one additional user in the historical data has not been evaluated. If so, the method 400 returns to block 410. If not (e.g., if all users reflected in the historical data have been evaluated), the method 400 terminates at block 435.

In some embodiments, the method 400 can be used periodically or upon other specified criteria (e.g., upon being triggered by a user or clinician) to generate or update training data used to train or refine one or more machine learning models. By iteratively using the method 400 (e.g., annually), the training data can be continuously or repeatedly updated, thereby allowing the machine learning system to refine the machine learning models to adjust to any changing conditions. This can significantly improve the accuracy and efficiency of such models.

### Example Method for Training Machine Learning Models to Evaluate User Depression

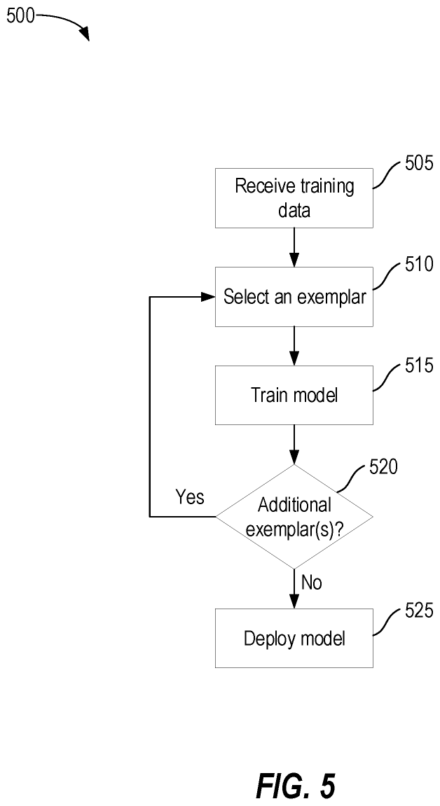

FIG. 5 is a flow diagram depicting an example method 500 for training machine learning models to evaluate user depression. In the illustrated embodiment, the method 500 is performed by a machine learning system, such as the machine learning system 135 of FIG. 1. In other embodiments, the method 500 can be performed by other systems, such as dedicated training systems.

The method 500 begins at block 505, where the machine learning system receives training data (e.g., the training data generated and/or updated at block 425 of FIG. 4). In embodiments, receiving the training data may include receiving it from one or more other systems (e.g., data aggregation or preprocessing systems), retrieving it from local or remote storage, and the like. In one embodiment, the training data can generally include multiple sets of user attributes, where each set of user attributes indicates the attributes or characteristics of a user during a window of time immediately preceding a diagnosis of one or more depressive disorders (e.g., within a defined window, such as one month prior, six months prior, and the like). In some embodiments, as discussed above, the training data may also include one or more sets of attributes that did not immediately-precede a diagnosis of one or more depressive disorders (e.g., to be used as negative exemplars in training the model).

At block 510, the machine learning system selects one of the exemplars included in the training data. As used herein, an exemplar refers to a set of attributes (e.g., corresponding to a defined or learned set of features) from a single user during a defined window of time. For example, the exemplar may include indications as to the demographics of the user, whether the user had any specified diagnoses at the time, whether the user had been clinically assessed with one or more defined conditions during the window, medication(s) the user used or was prescribed during the window, one or more natural language notes authored by or describing the user during the window, and the like.

Generally, the exemplar may be selected using any suitable criteria (including randomly or pseudo-randomly), as the machine learning system will use all exemplars during the training process. Although the illustrated example depicts selecting exemplars sequentially for conceptual clarity, in embodiments, the machine learning system may select and/or process multiple exemplars in parallel.

At block 515, the machine learning system trains the machine learning model based on the selected exemplar. For example, the machine learning system may use the attributes indicated in the exemplar to generate an output risk score or classification for the user. As discussed above, this risk score can generally indicate the probability that the user either currently has one or more depressive disorders, or will develop or be diagnosed with one or more depressive disorders in the near future (e.g., within a defined window, which may match to the window used to define the attributes). In one such embodiment, lower values may indicate a lower probability that the user has or will suffer from depression imminently, while a higher value indicates that the user is relatively more likely to currently have depression, or is more likely to suffer from depression imminently.

During training, this score can then be compared against a ground-truth associated with the selected exemplar (e.g., an indication as to whether the user had or developed depression during or after the time associated with the attributes). In some embodiments, this comparison includes determining how much time elapsed between the attribute(s) and the diagnosis of depression. Based on this comparison, the parameters of the machine learning model can be updated. For example, if the generated risk score is relatively low but the user was, in fact, diagnosed with a depressive disorder, the machine learning system may modify the parameters such that the attributes in the exemplar result in a larger risk score being generated.

At block 520, the machine learning system determines whether at least one additional exemplar remains in the training data. If so, the method 500 returns to block 510. If not, the method 500 continues to block 525. Although the illustrated example depicts iteratively refining the model using individual exemplars (e.g., using stochastic gradient descent), in some embodiments, the machine learning system can refine the model based on multiple exemplars simultaneously (e.g., using batch gradient descent).

At block 525, the machine learning system deploys the trained machine learning model for runtime use. In embodiments, this may include deploying the model locally (e.g., for runtime use by the machine learning system) and/or to one or more remote systems. For example, the machine learning system may distribute the trained model to one or more downstream systems, each responsible for one or more residential facilities (e.g., to one or more servers associated with specific care facilities, where these servers may use the model to evaluate depression risk for residents at the specific facility).

### Example Method for Using Trained Machine Learning Models to Generate Risk Scores

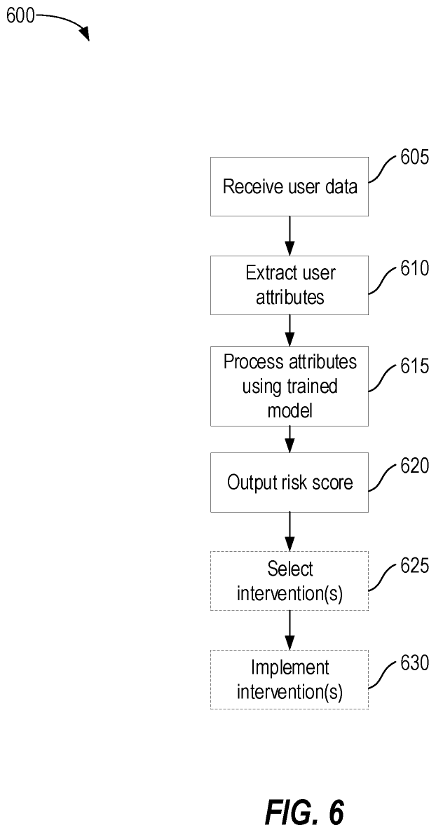

FIG. 6 is a flow diagram depicting an example method 600 for using trained machine learning models to use machine learning to generate risk scores and implement appropriate interventions. In the illustrated embodiment, the method 600 is performed by a machine learning system, such as the machine learning system 135 of FIG. 2. In other embodiments, the method 600 can be performed by other systems, such as dedicated inferencing systems.

At block 605, the machine learning system receives user data (e.g., user data 205 of FIG. 2). As discussed above, the user data may include data or information associated with a user (also referred to as a patient or a resident in some aspects). In some embodiments, the user data includes attributes for a resident residing in a long-term care facility.

In an embodiment, the user data can generally include information relating to attributes of the user, such as demographics of the user, a set of one or more diagnoses of the user, medications used by the user, clinical assessments of condition(s) of the user, one or more natural language notes authored by or describing the user, and the like. In some embodiments, the received user data corresponds to current information for the user. That is, the user data may be the most-recent data for each feature.

In at least one aspect, the user data is received because a change has occurred. That is, the user data may be provided to the machine learning system (e.g., using a push technique) based on determining that one or more of the attributes have changed since the last time the data was provided. In other embodiments, the machine learning system can retrieve or request the user data, and evaluate it to determine whether any attributes have changed. In at least one embodiment, if no attributes have changed (with respect to the relevant features used by the model), the machine learning system can refrain from further processing of the data (e.g., refrain from generating a new risk score), thereby reducing computational expense.

Similarly, if the data is only provided upon detecting a change, the machine learning system need not review it at all, which also reduces computational expense of the system. Additionally, in some embodiments, the machine learning system can receive only the updated data (as opposed to receiving or retrieving the entirety of the user's data). That is, the storage systems may automatically transmit data when it is updated (or the machine learning system may request any new or changed data), enabling the risk score to be revised based on the new data without the need to re-transmit the older data. This, again, reduces the computational expense (including bandwidth, if the data is stored remotely from the machine learning system) of generating the scores.

In some embodiments, the received user data includes data corresponding to a defined set of features that are used by the machine learning model. These features may include, for example, specific demographic data, specified diagnoses, specified clinical assessments and/or conditions, specified medications, and the like. In some embodiments, the user data can include additional data beyond these features (e.g., information about all medications that the resident has been prescribed, regardless of the specific medications used in the machine learning model). In one such embodiment, the machine learning system can identify and extract the relevant attributes or data, based on the indicated features for the model. In other embodiments, the received user data may include only the specific data corresponding to the indicated features (e.g., another system may filter the user data based on the features, thereby protecting data that is not needed to build the model). In still another aspect, such unused features or attributes may simply be associated with a weight of zero in the model.

At block 610, the machine learning system extracts the set of relevant user attributes, from the user data, based on the specified features that are used by the machine learning model. That is, the machine learning system can extract, from the user data, the relevant information for each feature. For example, if a specific diagnosis is included in the features, the machine learning system may search the user data using one or more diagnosis codes (e.g., ICD-10 codes) corresponding to the specific diagnosis. If the user currently has the diagnosis, it can be indicated in the corresponding user attribute (e.g., with a value indicating the presence of the diagnosis). If the diagnosis is not found, this attribute may be set to a defined value, such as a null value or a value of zero, to indicate that the user does not have the feature.

At block 615, the machine learning system processes the identified/extracted attributes using a trained machine learning model. As discussed above, the machine learning model may generally specify a set of parameters (e.g., weights) for input features and/or intermediate features (within internal portions of the model) learned during training. In some embodiments, as discussed above, the model may specify weights for individual features, for groups of features, or for both individual features and groups of features. For example, a “diagnosis” category may be assigned a first weight. To generate a score for the diagnosis category, the individual diagnoses specified within may each be associated with respective weights. That is, a diagnosis of one disorder may have a higher weight than a second disorder.

In this way, the machine learning system can use the weights to generate a score for each group of features (if groups are used), such as by multiplying the weight of each feature with an indication as to whether the user has the feature (or, in some aspects, a value indicating the severity of the feature). For example, in some embodiments, the machine learning system can use binary indications, where the user either has the feature (e.g., a value of one) or does not (e.g., a value of zero). In some embodiments, the machine learning system can additionally or alternatively use non-binary values to indicate the severity (e.g., where a value of zero may indicate that the user does not have the feature, a value of one indicates that the resident has a mild case, a value of two indicates a moderate case, and so on).

The resulting values for each weighted attribute can then be combined (e.g., summed) to generate an overall score with respect to the feature group. In an embodiment, the machine learning system can similarly generate scores for each other group, and then aggregate these (potentially using a weighted sum using learned weights, as discussed above) to generate an overall risk score for the resident.

At block 620, the machine learning system outputs the generated risk score. In embodiments, this may include, for example, outputting it via a display or terminal (e.g., for a caregiver to review). In some embodiments, block 620 includes outputting the risk score to one or more other systems (e.g., a storage repository, or other systems that evaluate the risk information to inform allocation or intervention decisions), and the like.

At block 625, the machine learning system (or another system, such as the intervention system 240 of FIG. 2) can optionally select one or more interventions based on the generated risk score. That is, as discussed above, the system may select one or more prophylactic interventions to reduce potential harm, based on the current depression risk. For example, based on an increase in risk, the system may allocate or suggest additional resources to the resident (such as additional therapy sessions), instruct a user (e.g., a caregiver) to care for the resident, prescribe or suggest medications for the user, and the like. In some embodiments, the system can use other specific and targeted non-medical interventions for the specific user. For example, the system may retrieve audio (e.g., a song that the user likes, or a voice recording from a loved one of the user).

In at least one embodiment, the system can determine whether to select interventions based at least in part on whether the risk score for the user has changed (e.g., since the last score, in the last 24 hours, and the like), based on the magnitude of the change, based on the direction of the change, based on the value of the current score, and the like. For example, the machine learning system may determine to generate an alert based on determining that the risk score has increased, and the increase exceeds a threshold percentage and/or exceeds a threshold value.

At block 630, the system optionally implements the selected intervention(s). This may include a wide variety of actions, including revised resource allocations, outputting audio or other media, prescribing medications, and the like.

### Example Method for Extracting User Attributes for Machine Learning

FIG. 7 is a flow diagram depicting an example method 700 for extracting user attributes for input to machine learning models. In the illustrated embodiment, the method 700 is performed by a machine learning system, such as the machine learning system 135 of FIGS. 1 and/or 2. In other embodiments, the method 700 can be performed by other systems, such as dedicated training or inferencing systems. In some embodiments, the method 700 provides additional detail for block 420 of FIG. 4 and/or block 610 of FIG. 6.

At block 705, the machine learning system extracts natural language input for the model. For example, the machine learning system may identify and evaluate natural language notes (e.g., natural language notes 130 in FIG. 1, and/or natural language notes 230 in FIG. 2). In some embodiments, as discussed above, the natural language input can generally correspond to written notes (e.g., authored by a clinician) about the user (e.g., describing their demeanor, condition, and the like). In some embodiments, the natural language input can include verbal or recorded notes. For example, a clinician may record themselves speaking about (or with) the user. In one such embodiment, one or more speech-to-text techniques can be used to transcribe the note for processing.

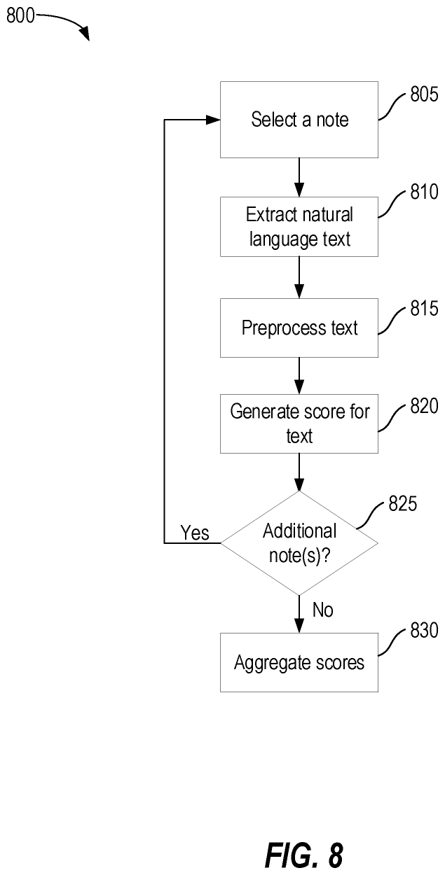

In at least one embodiment, the machine learning system can perform one or more preprocessing operations on the natural language text to extract the input. For example, as discussed above with reference to FIG. 3, the machine learning system may extract the text itself, normalize it, remove noise and/or redundant elements, lemmatize it, tokenize it, generate one or more roots for it, vectorize it, and the like. One example for extracting and evaluating the natural language input is described in more detail below with reference to FIG. 8.

At block 710, the machine learning system extracts demographics for the user. In some embodiments, as discussed above, the user demographics can generally include features such as their age, gender, marital status (and, in some embodiments, whether the marital status recently changed, such as within a defined window of time), race, and the like. In some embodiments, the particular features reflected by the extracted demographics are determined based on the features used by the machine learning model. As discussed above, in some embodiments, these features are manually defined or curated. In other embodiments, the machine learning system can use feature selection techniques to automatically identify salient features.

At block 715, the machine learning system extracts diagnoses for the user. In an embodiment, the diagnoses input can generally indicate whether the user has one or more specified diagnoses. In some embodiments, as discussed above, these diagnoses may be from a defined set of diagnoses selected due to their salience to depression risk (e.g., by a user, or automatically using machine learning or feature extraction). In some embodiments, the machine learning system can also determine the severity of the diagnosis. In some embodiments, in addition to identifying current diagnoses, the machine learning system can also determine the recency of one or more diagnoses. That is, the machine learning system may use not only the presence of a diagnosis as an input feature, but also use the severity as another input feature, and further use how recently that diagnosis was received as an input feature. For example, higher severity may be processed with higher weight, and more recent diagnoses may be treated with increased weight.

At block 720, the machine learning system extracts one or more clinical assessments for the user. As discussed above, the clinical assessments can generally correspond to assessments or evaluations of the user's functional state (e.g., prepared by a clinician). For example, the extracting the assessments may include determining whether the user has recently been assessed for weight loss, weight gain, pain, increased or decreased food intake, isolation (either social, such as due to limited family, or physical, such as due to illness), one or more mood or behavioral issues, and the like. In embodiments, the particular assessments that are extracted may be determined based on the features used by the machine learning model, which may be defined manually and/or using machine learning or feature selection techniques. In some embodiments, in addition to evaluating which assessments are present, the machine learning system can also consider the recency of the assessments. For example, in addition to or instead of simply determining whether the user has been assessed with weight loss, the machine learning system may determine how recently the weight loss occurred, and use this recency as input to the model. For example, more recent assessments may be treated with increased weight.

At block 725, the machine learning system extracts medications for the user. In an embodiment, the medication input can generally indicate whether the user has been prescribed (or receives) one or more specified medications. In some embodiments, as discussed above, these medications may be from a defined set selected due to their salience to depression risk (e.g., by a user, or automatically using machine learning or feature extraction). In some embodiments, the machine learning system can also determine not only the medication(s) received, but also the dosage. In some embodiments, in addition to identifying current medications, the machine learning system can also determine the recency of the prescription. That is, the machine learning system may use not only the use of a given medication as an input feature, but also use the prescribed dosage, as well as how recently that prescription was received or updated. For example, higher dosages may be processed with higher weights, and more recent prescriptions may be treated with increased weight.

Using the method 700, the machine learning system can therefore extract relevant user attributes to train machine learning model(s) to predict depression risk, or to be used as input to trained models in order to generate predicted risks during runtime.

### Example Method for Evaluating Unstructured Data for Machine Learning

FIG. 8 is a flow diagram depicting an example method 800 for evaluating unstructured input data to improve machine learning. In the illustrated embodiment, the method 800 is performed by a machine learning system, such as the machine learning system 135 of FIGS. 1 and/or 2. In other embodiments, the method 800 can be performed by other systems, such as dedicated natural language processing systems. In some embodiments, the method 800 provides additional detail for block 705 of FIG. 7.

At block 805, the machine learning system can select one of the notes included in the user data. As discussed above, these notes are generally associated with the user, and can include notes written by the user (e.g., describing their own condition or feelings, or describing an unrelated topic), notes describing the user, written by another individual (e.g., clinical notes prepared by a clinician during or immediately after a healthcare visit with the user), and the like. In some embodiments, the note is selected from a subset of notes that were recorded within a defined window of time, such as within three months of the index date (e.g., the date for which the depression risk is being generated). Generally, the note may be selected using any suitable technique, as all relevant notes (e.g., all notes recorded within the defined window) will be evaluated in turn. Although an iterative process is depicted for conceptual clarity, in some aspects, the machine learning system can evaluate some or all of the notes in parallel.

At block 810, the machine learning system can extract natural language text from an unstructured portion of the selected note. For example, the note may include one or more structured elements (such as a name field, a date field, and the like), as well as one or more unstructured portions (e.g., the portion that the author uses to write their assessments or thoughts).

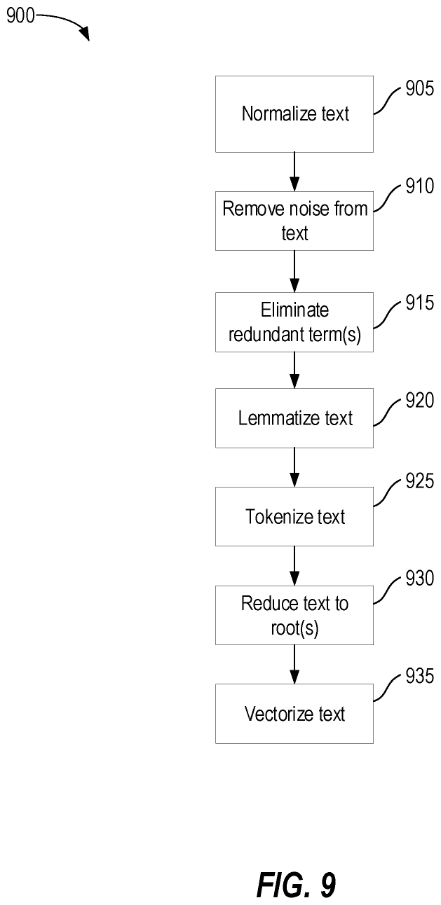

At block 815, the machine learning system can optionally preprocess the extracted natural language text. For example, as discussed above with reference to FIG. 3, the machine learning system may normalize the text, remove noise and/or redundant elements, lemmatize it, tokenize it, generate one or more roots for it, vectorize it, and the like. One example for preprocessing the natural language text is described in more detail below with reference to FIG. 9.

At block 820, the machine learning system generates a score for the extracted (and, in some embodiments, preprocessed) text. The note score may generally correspond to whether the note indicates depression, where higher values indicate an increased probability that the note reflects depressive disorder (or a more severe depressive disorder). In some embodiments, the machine learning system generates the score by processing the text using one or more trained machine learning models.

For example, the machine learning system may train a machine learning model that generates the score based on the extracted natural language text (e.g., using labeled exemplars, such as prior notes that have a corresponding score assigned by a user). That is, a machine learning model may be trained by using prior notes as input, with a user-defined depression score as target output. Once trained, the model may be used to generate new scores for newly-received notes. These scores, as discussed below in more detail, can then be used as input to the overall depression model.

In some embodiments, the machine learning system uses sentiment analysis techniques to score the note. In some embodiments, the machine learning system can use various natural language techniques, such as keyword matching. For example, the machine learning system may determine whether any defined keywords that relate to depression are found in the text, determine how many of the keywords are found, determine a relative frequency of such keywords (as compared to the overall length of the text), and the like. In one such embodiment, the score can indicate whether such keywords were found, the number and/or frequency of the keywords, and the like.

At block 825, the machine learning system determines whether there is at least one additional note that has not been processed. If so, the method 800 returns to block 805. If all of the relevant notes have been evaluated and scored, the method 800 continues to block 830.

At block 830, the machine learning system aggregates the scores that were generated for each note. For example, the machine learning system may sum the scores, average the scores, and the like. In at least one embodiment, the machine learning system determines the number of notes having a score that exceeds one or more thresholds, and uses this number as the aggregate score for the notes. For example, the machine learning system may determine that the average note score was a specific value (such as 72), determine that six notes exceeded some threshold value, determine that some percentage of the notes (e.g., 60%) exceeded the threshold, and the like.

This aggregate information can then be used as the natural language input to the machine learning model. That is, the average note score, percentage or number of notes exceeding a threshold score, and the like may be used as a “natural language” feature to be used as input to the model, in order to generate a depression risk score.

### Example Method for Preprocessing Unstructured Data to Improve Machine Learning

FIG. 9 is a flow diagram depicting an example method 900 for preprocessing unstructured input data to improve machine learning results. In the illustrated embodiment, the method 900 is performed by a machine learning system, such as the machine learning system 135 of FIGS. 1 and/or 2. In other embodiments, the method 900 can be performed by other systems, such as dedicated natural language processing systems or preprocessing systems. In some embodiments, the method 900 provides additional detail for the workflow 300 of FIG. 3, and/or for block 815 in FIG. 8.

Generally, each block in the method 900 is optional, and the machine learning system may perform all of the indicated operations, or some subset thereof. The machine learning system may also use additional preprocessing steps not depicted in the illustrated example. Additionally, though the illustrated example suggests a linear and sequential process for conceptual clarity, in embodiments, the operations may be performed in any order (including entirely or partially in parallel).

At block 905, the machine learning system can normalize the extracted natural language text. As discussed above, this normalization may include a wide variety of text normalization processes, such as converting all characters in the extracted text to lowercase, converting accented or foreign language characters to ASCII characters, expanding contractions, converting words to numeric form where applicable, converting dates to a standard date format, and the like.

At block 910, the machine learning system removes noise from the text. As discussed above, noise removal may include identification and removal of portions of the extracted text that do not carry meaningful or probative value, such as characters, portions, or elements of the text that are not useful or meaningful in the ultimate computing task (e.g., computing a risk score), and/or that are not useful to human readers. For example, the noise removal may include removing extra white or blank spaces, tabs, or lines, removing tags such as HTML tags, and the like.

At block 915, the machine learning system can eliminate redundant elements or terms from the text. As discussed above, this may include identifying and eliminating or removing text corresponding to redundant elements (e.g., duplicate words), and/or the reduction of a sentence or phrase to a portion thereof that is most suitable for machine learning training or application. For example, the redundancy elimination may include eliminating verbs (which may be unhelpful in the machine learning task), conjunctions, or other extraneous words that do not aid the machine learning task.

At block 920, the machine learning system lemmatizes the text. As discussed above, text lemmatization can generally include stemming and/or lemmatization of one or more words in the extracted text. This may include converting words from their inflectional or other form to a base form. For example, lemmatization may include replacing “holding,” “holds,” and “held” with the base form “hold.”

At block 925, the machine learning system tokenizes the text. In an embodiment, tokenizing the text may include transforming or splitting elements in the extracted text (e.g., strings of characters) into smaller elements, also referred to as “tokens.” For example, the tokenization may include tokenizing a paragraph into a set of sentences, tokenizing a sentence into a set of words, transforming a word into a set of characters, and the like. In some embodiments, tokenization can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

At block 930, the machine learning system can reduce the text to one or more roots. As discussed above, the root generation can include reducing portion of the extracted text (e.g., a phrase or sentence) to its most relevant n-gram (e.g., a bigram) or root for downstream machine learning training and/or application.

At block 935, the machine learning system can vectorize the text. Generally, vectorization may include converting the text into one or more objects that can be represented numerically (e.g., into a vector or tensor form). For example, the machine learning system may use one-hot encodings (e.g., where each element in the vector indicates the presence or absence of a given word, phrase, sentiment, or other concept, based on the value of the element). In some embodiments, the machine learning system can generate one or more word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate depression risk scores).

### Example Method for Using Machine Learning to Evaluate Trends in User Risk

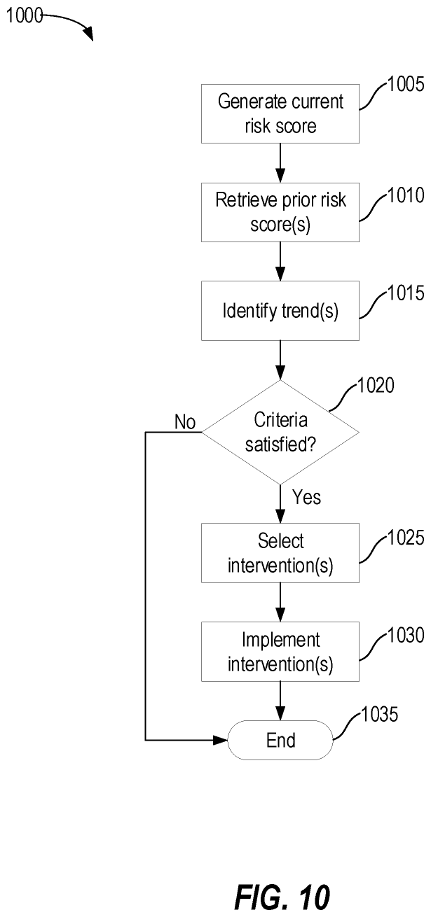

FIG. 10 is a flow diagram depicting an example method 1000 for using machine learning to evaluate trends in user risk and implement appropriate interventions. In the illustrated embodiment, the method 1000 is performed by a machine learning system, such as the machine learning system 135 of FIG. 2. In other embodiments, the method 1000 can be performed by other systems, such as dedicated inferencing systems. In some embodiments, the method 1000 may be used to provide more long-term analysis and interventions, as compared to the method 600 of FIG. 6.

At block 1005, the machine learning system generates a current depression risk score for a user based on a set of current attributes. For example, as discussed above with reference to FIGS. 2 and 6, the machine learning system may extract and process the relevant attributes using one or more trained machine learning models. In some embodiments, the risk score is considered “current” because it is generated using attributes that are within a defined window from the current time (e.g., within the last month, the last three months, the last six months, the last year, and the like).

At block 1010, the machine learning system retrieves one or more prior risk scores that have been generated for the user. This may include generating risk scores based on prior data (e.g., based on a set of attributes from a prior window), as well as retrieving one or more risk scores that were previously-generated and stored.

At block 1015, the machine learning system can evaluate the current and prior risk score(s) to identify any patterns or trends. For example, the machine learning system may use regression to determine whether the risk scores are stable, trending upward, trending downward, and the like. In some embodiments, the machine learning system can additionally or alternatively identify variations or patterns such as seasonal trends (e.g., where the score tends to increase in the winter and decrease in the summer), or determining whether the score changes generally correlate with other external factors (such as the passing of a loved one, dissolution of a marriage, and the like).

At block 1020, the machine learning system determines whether one or more criteria are satisfied. In some embodiments, this can include determining whether any trends or patterns were identified. In at least one embodiment, the criteria relate to whether the trend(s) indicate that the depression score is currently increasing and/or is likely to increase in the future. For example, if the scores are trending upwards, the machine learning system can determine that the criteria are satisfied. As another example, if the machine learning system determines that the scores fluctuate with the seasons, and that the upcoming season will likely cause it to increase, the machine learning system can determine the criteria are satisfied.

If the criteria are not satisfied (e.g., because no trends are identified, or because the identified trends indicate that the depression risk scores are decreasing or will decrease), the method 1000 terminates at block 1035. If the criteria are satisfied, the method 1000 continues to block 1025.

At block 1025, the machine learning system selects one or more interventions for the user. In some embodiments, the intervention(s) can be selected based at least in part on the identified trends. For example, if the trend indicates that the risk score is correlated with external events related to grief, the machine learning system may select one or more grief counseling interventions. As another example, if the trend indicates that the risk score varies seasonally, the machine learning system may select interventions that can reduce these seasonal patterns (e.g., suggesting specific indoor activities during colder months).

At block 1030, the machine learning system can then implement the intervention(s). In embodiments, the processes used to implement the interventions may vary depending on the type of intervention. For example, some interventions may be implemented by generating and outputting an alert or suggestion (e.g., to the user or a clinician). The method 1000 then terminates at block 1035.

### Example Method for Using Machine Learning to Evaluate and Aggregate Risk

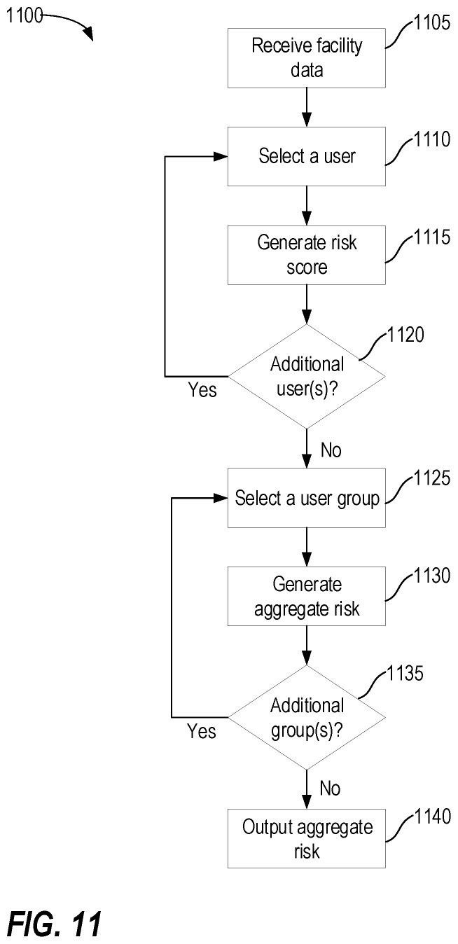

FIG. 11 is a flow diagram depicting an example method 1100 for using machine learning to evaluate and aggregate user risk. In the illustrated embodiment, the method 1100 is performed by a machine learning system, such as the machine learning system 135 of FIG. 2. In other embodiments, the method 1100 can be performed by other systems, such as dedicated inferencing systems.

At block 1105, the machine learning system receives facility data. In an embodiment, the facility data can generally include data relating to the residents of a given residential care facility. For example, in at least one aspect, the facility data can include, for each resident in the facility, a corresponding resident profile (e.g., including the user data 205, in FIG. 2). As discussed above, this data can generally reflect attributes or characteristics of the residents, such as their diagnoses, assessments, medications, and the like.

At block 1110, the machine learning system selects one of the users reflected in the facility data. Generally, this selection can be performed using any technique (including randomly or pseudo-randomly), as the machine learning system will evaluate all of the facility's residents during the method 1100. Although an iterative or sequential process is depicted for conceptual clarity, in some embodiments, the machine learning system may evaluate some or all of the user data in parallel.

At block 1115, the machine learning system generates a depression risk score for the selected user. For example, using the workflow 200 of FIG. 2 and/or the method 600 of FIG. 6, the machine learning system may process the attributes of the selected user using the trained machine learning model(s).

At block 1120, the machine learning system determines whether there is at least one additional user, resident in the facility, that has not-yet been evaluated. In some embodiments, this can include determining whether there are any users with an out-of-date risk score. That is, the machine learning system can determine whether any of the remaining users have updated data that has not been used to generate a risk score, as discussed above. If so, the method 1100 can return to block 1110. If an updated risk score has been generated for all of the users in the facility, the method 1100 continues to block 1125.

At block 1125, the machine learning system selects a group of users. In embodiments, these user groups may be defined according to a wide variety of criteria, depending on the particular implementation. In at least one embodiment, the user groups correspond to the physical distribution of residents in the facility. For example, the user groups may correspond to separate wings, buildings, and the like. This can allow the machine learning system to evaluate resident depression risk on a per-region or per-locale basis, enabling improved resource allocation and intervention. In some embodiments, the user groups are defined based on staffing assignments. For example, each user group may correspond to a given caregiver (e.g., all residents primarily assigned to that caregiver), enabling the machine learning system to evaluate resident depression risks on a per-caregiver basis, which may help in balancing the workload of each caregiver and prevent or reduce mistakes or other negative outcomes caused by an overloaded caregiver.

At block 1130, the machine learning system generates an aggregated depression risk for the selected group. In some embodiments, the aggregated risk can correspond to the average risk score of the group's members. In some embodiments, the aggregate risk is the sum of the individual risks of each resident in the group. In at least one embodiment, the aggregate risk can indicate the distribution of depression risks (e.g., based on classes or categories of risk), as discussed above.

At block 1135, the machine learning system determines whether there is at least one additional user group that has not been evaluated. If so, the method 1100 returns to block 1125. If not, the method 1100 proceeds to block 1140.

At block 1140, the machine learning system can output the aggregated risk scores. For example, as discussed above, the machine learning system may output the aggregate data on a GUI, enabling users to quickly review how the depression risks are distributed across the facility, across caregivers, and the like. In some embodiments, outputting the aggregate risk can include outputting it to one or more downstream systems that can select and/or implement various interventions, as discussed above.

### Example Method for Refining Machine Learning Models

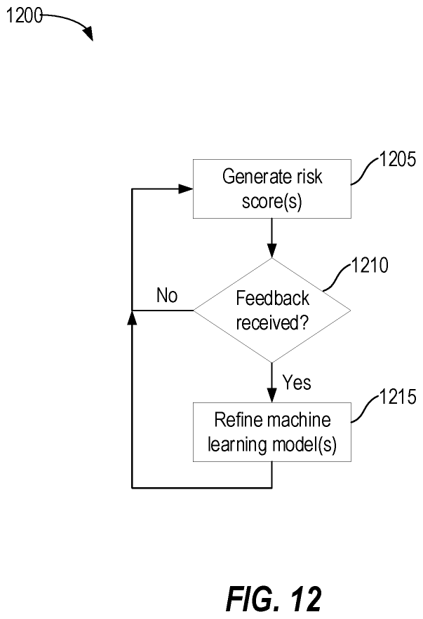

FIG. 12 is a flow diagram depicting an example method 1200 for refining machine learning models based on user risk. In the illustrated embodiment, the method 1200 is performed by a machine learning system, such as the machine learning system 135 of FIGS. 1 and/or 2. In other embodiments, the method 1200 can be performed by other systems, such as dedicated training or refinement systems.

At block 1205, the machine learning system can generate one or more risk scores during runtime. That is, the machine learning system may deploy and use a trained machine learning model to process user data in order to generate depression risk scores at runtime. For example, the machine learning system may automatically use the model(s) to generate risk scores for all residents in a residential care facility, for all patients served by a clinician, and the like. Similarly, the machine learning system may automatically generate new scores whenever new data becomes available. In some embodiments, a clinician can selectively use the machine learning system to generate risk scores as needed.

At block 1210, the machine learning system determines whether any feedback relating to the generated risk scores has been received. In some embodiments, this feedback is manual or explicit. In one such embodiment, a clinician or user may indicate whether one or more generated risk scores are accurate. For example, the user may indicate that the machine learning system generated a relatively low score for a user that has (or subsequently developed) depression, that the machine learning system generated a relatively high score for a user that does not have (and did not develop) depression, and the like. In some embodiments, the user may alternatively or additionally indicate that the score was accurate.

In at least one embodiment, the feedback can be automatically determined. In one such embodiment, after generating a risk score for a user, the machine learning system may continuously or periodically evaluate the user's data to determine whether they were diagnosed or assessed with depression (e.g., based on whether one or more depressive disorder codes were entered into the user's EHR). In at least one embodiment, the machine learning system can perform this analysis the next time a risk score is generated for the user. That is, each time the machine learning system generates a risk score for the user, the machine learning system can determine whether the user has been diagnosed with depression in the interim since the time when the last risk score was generated.

At block 1215, the machine learning system refines the trained machine learning model(s) based on this feedback. For example, if the generated risk score for a user was relatively low, but the feedback indicates that the user developed depression, the machine learning system may use the prior set of attributes (used to generate the score) as exemplar input that should be correlated with an increased risk of depression. Similarly, if the generated risk score was relatively high but the feedback indicates the user does not have depression, the machine learning system may refine the model to indicate that such attributes should be correlated to a lower risk.

In at least one embodiment, the machine learning system may additionally or alternatively query whether one or more events or other factors occurred that might explain the change. For example, if the user recently suffered an unexpected loss, the machine learning system may refrain from revising the model, as this loss may explain the recent depression, and a prior-generated low score may have been accurate at the time. Similarly, if the user recently experienced a positive event (e.g., winning the lottery), the machine learning system may again refrain from revising the model, as a lack of depression may be explained at least in part on these good events, and a prior-generated high score may have been accurate at the time.

The method 1200 then returns to block 1205. In this way, the machine learning system can continuously or periodically refine the machine learning model(s), thereby ensuring that they continue to produce highly-accurate risk score predictions for users.

### Example Method for Training Machine Learning Models to Improve Risk Evaluation

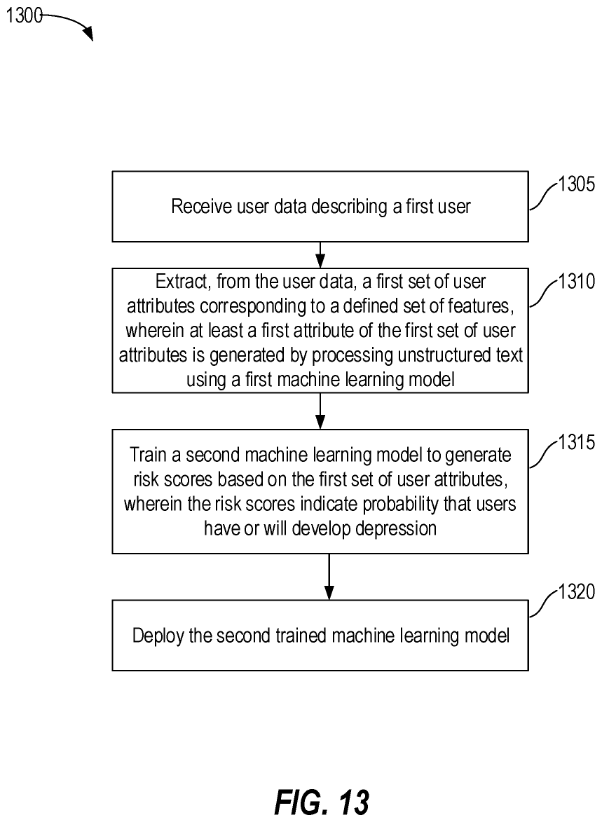

FIG. 13 is a flow diagram depicting an example method 1300 for training machine learning models to improve user risk evaluation. In the illustrated embodiment, the method 1300 is performed by a machine learning system, such as the machine learning system 135 of FIG. 1. In other embodiments, the method 1300 can be performed by other systems, such as dedicated training or refinement systems.

At block 1305, user data (e.g., historical data 105 of FIG. 1) describing a first user is received.

At block 1310, a first set of user attributes (e.g., diagnoses 110, medications 115, clinical assessments 120, demographics 125, and/or natural language notes 130, each of FIG. 1) corresponding to a defined set of features is extracted from the user data, wherein at least depression first attribute of the first set of user attributes is generated by processing unstructured text using a first trained machine learning model.

At block 1315, a second machine learning model (e.g., machine learning model 140 of FIG. 1) is trained to generate risk scores based on the first set of user attributes, wherein the risk scores indicate probability that users have or will develop depression.

At block 1320, the second trained machine learning model is deployed.

### Example Method for Generating Risk Scores Using Machine Learning

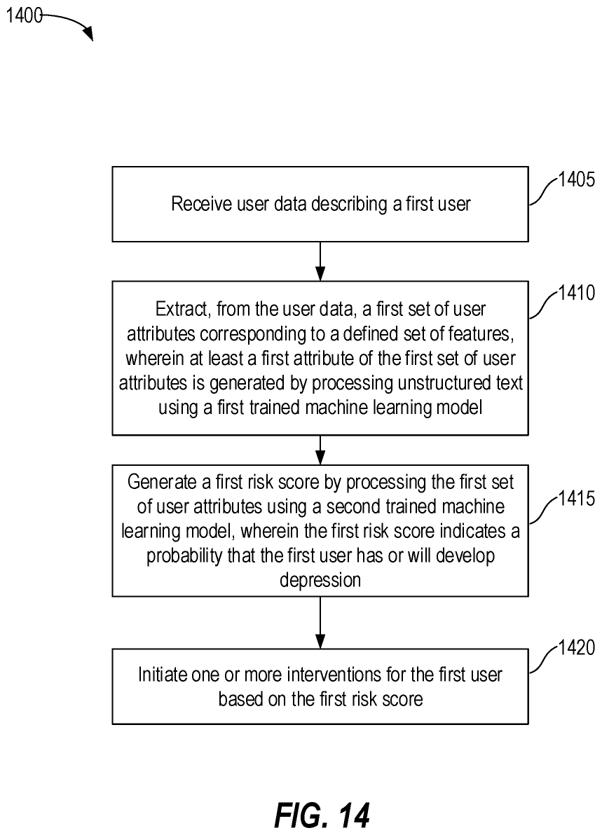

FIG. 14 is a flow diagram depicting an example method 1400 for generating risk scores using trained machine learning models. In the illustrated embodiment, the method 1400 is performed by a machine learning system, such as the machine learning system 135 of FIG. 2. In other embodiments, the method 1400 can be performed by other systems, such as dedicated inferencing systems.

At block 1405, user data (e.g., user data 205 of FIG. 2) describing a first user is received.

At block 1410, a first set of user attributes (e.g., diagnoses 210, medications 215, clinical assessments 220, demographics 225, and/or natural language notes 230, each of FIG. 2) corresponding to a defined set of features is extracted from the user data, wherein at least a first attribute of the first set of user attributes is generated by processing unstructured text using a first trained machine learning model.

At block 1415, a first risk score (e.g., risk score 235 of FIG. 2) is generated by processing the first set of user attributes using a second trained machine learning model (e.g., machine learning model 140 of FIG. 1), wherein the first risk score indicates a probability that the first user has or will develop depression.

At block 1420, one or more interventions (e.g., interventions 245 of FIG. 2) for the first user are initiated based on the first risk score.

### Example Processing System for Improved Machine Learning

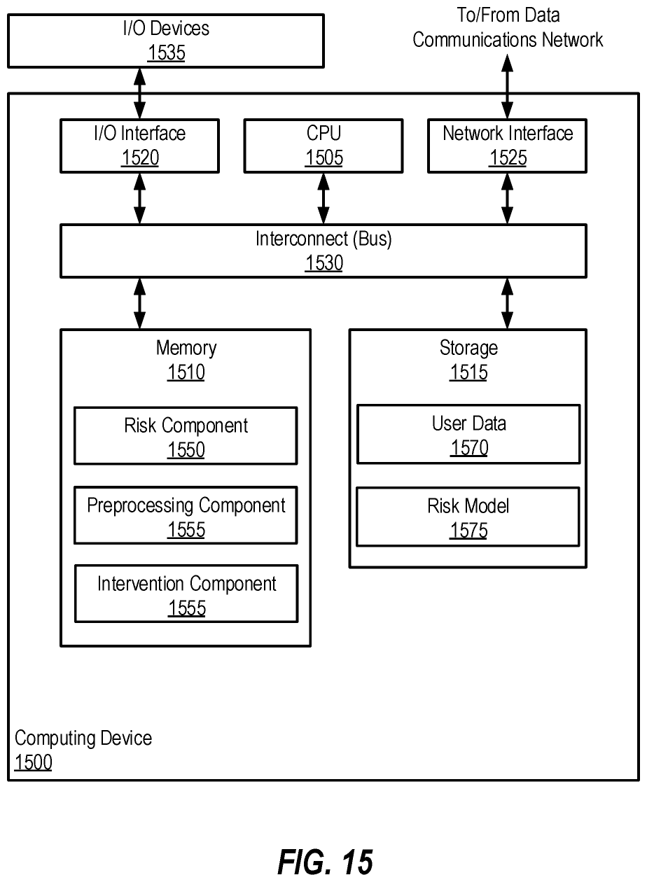

FIG. 15 depicts an example computing device 1500 configured to perform various aspects of the present disclosure. Although depicted as a physical device, in embodiments, the computing device 1500 may be implemented using virtual device(s), and/or across a number of devices (e.g., in a cloud environment). In one embodiment, the computing device 1500 corresponds to the machine learning system 135 of FIG. 1 and FIG. 2.

As illustrated, the computing device 1500 includes a CPU 1505, memory 1510, storage 1515, a network interface 1525, and one or more I/O interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes a risk component 1550, a preprocessing component 1555, and an intervention component 1560, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

In one embodiment, the risk component 1550 is used to generate input data, to train machine learning models (e.g., based on historical data), and/or to generate risk scores using trained models, as discussed above. The preprocessing component 1555 may generally be used to perform any needed or desired preprocessing on the data, such as text preprocessing (e.g., including normalization). The intervention component 1560 may be configured to use the generated risk scores to select, generate, and/or implement various interventions (such as alerts, prophylactic and specific/personalized interventions, and the like), as discussed above.

In the illustrated example, the storage 1515 includes user data 1570 (which may correspond to historical data, such as historical data 105 of FIG. 1, and/or to current user data, such as user data 205 of FIG. 2), as well as an risk model 1575 (which may correspond to the machine learning model 140 of FIG. 1). Although depicted as residing in storage 1515, the user data 1570 and risk model 1575 may be stored in any suitable location, including memory 1510.

### Example Clauses

Implementation examples are described in the following numbered clauses:

Clause 1: A method, comprising: receiving user data describing a first user; extracting, from the user data, a first set of user attributes corresponding to a defined set of features, wherein at least a first attribute of the first set of user attributes is generated by processing unstructured text using a first machine learning model; training a second machine learning model to generate risk scores based on the first set of user attributes, wherein the risk scores indicate probability that users have or will develop depression; and deploying the second trained machine learning model.

Clause 2: The method of Clause 1, wherein extracting the first set of user attributes comprises: identifying a plurality of notes in the user data comprising natural language text describing the first user; generating a set of scores by processing the plurality of notes using the first trained machine learning model; and aggregating the set of scores to generate the first attribute.

Clause 3: The method of any one of Clauses 1-2, wherein generating the set of scores further comprises preprocessing at least a first note of the plurality of notes, comprising: normalizing natural language text in the first note; and converting the normalized natural language text to a numerical vector.

Clause 4: The method of any one of Clauses 1-3, wherein training the second machine learning model further comprises: generating training data based on prior user data, the prior user data indicating a respective set of user attributes for each respective user of a plurality of users, comprising: identifying a set of records, in the prior user data, indicating that a corresponding user has depression, based on searching the prior user data for a defined medical code; and for each respective record in the set of records, extracting a respective set of user attributes corresponding to a defined window of time prior to a time associated with the respective record.

Clause 5: A method, comprising: receiving user data describing a first user; extracting, from the user data, a first set of user attributes corresponding to a defined set of features, wherein at least a first attribute of the first set of user attributes is generated by processing unstructured text using a first trained machine learning model; generating a first risk score by processing the first set of user attributes using a second trained machine learning model, wherein the first risk score indicates a probability that the first user has or will develop depression; and initiating one or more interventions for the first user based on the first risk score.

Clause 6: The method of Clause 5, wherein extracting the first set of user attributes comprises: identifying a plurality of notes in the user data, wherein each of the plurality of notes comprise natural language text describing the user; generating a set of scores for the plurality of notes by processing the plurality of notes using the first trained machine learning model; and aggregating the set of scores to generate the first attribute.

Clause 7: The method of any one of Clauses 5-6, wherein generating the set of scores by processing the plurality of notes further comprises preprocessing at least a first note of the plurality of notes, comprising: normalizing natural language text in the first note; and converting the normalized natural language text in the first note to a numerical vector.

Clause 8: The method of any one of Clauses 5-7, further comprising: determining that the first risk score exceeds a defined threshold; and generating an alert identifying the first user.

Clause 9: The method of any one of Clauses 5-8, further comprising: identifying a most impactful attribute, from the first set of user attributes, that caused the first risk score to exceed the defined threshold; and indicating the most impactful attribute in the generated alert.

Clause 10: The method of any one of Clauses 5-9, further comprising: for each respective user of a plurality of users in a healthcare facility: identifying a respective set of user attributes; and generating a respective risk score for the respective user by processing the respective set of user attributes using the second trained machine learning model.

Clause 11: The method of any one of Clauses 5-10, wherein the defined set of features comprises: one or more features relating to diagnoses, one or more features relating to clinical assessments, and one or more features relating to medications.

Clause 12: The method of any one of Clauses 5-11, wherein: the one or more features relating to diagnoses comprise a defined set of diagnoses, and the first set of user attributes indicates, for each respective diagnosis of the defined set of diagnoses, whether the first user has the respective diagnosis.

Clause 13: The method of any one of Clauses 5-12, wherein the first set of user attributes further indicates, for each respective diagnosis of the defined set of diagnoses, whether the first user was diagnosed with the respective diagnosis within a defined window of time.

Clause 14: The method of any one of Clauses 5-13, wherein: the one or more features relating to clinical assessments comprise a defined set of conditions, recorded by one or more caregivers, relating to functional states of users, and the first set of user attributes indicates, for each respective condition of the defined set of conditions, whether the first user has the respective condition.

Clause 15: The method of any one of Clauses 5-14, wherein the defined set of conditions comprises at least one of: (i) weight loss, (ii) weight gain, (iii) pain, (iv) increased food intake, (v) decreased food intake, (vi) isolation, or (vii) one or more mood or behavioral issues.

Clause 16: The method of any one of Clauses 5-15, wherein: the one or more features relating to medications comprise a defined set of medications, and the first set of user attributes indicates, for each respective medication of the defined set of medications, whether the first user receives the respective medication.

Clause 17: The method of any one of Clauses 5-16, wherein the first set of user attributes further indicates, for each respective medication of the defined set of medications, whether the first user was prescribed the respective medication within a defined window of time.

Clause 18: The method of any one of Clauses 5-17, wherein the second trained machine learning model was trained on prior user data for a plurality of users, the prior user data indicating a respective set of user attributes for each respective user of the plurality of users.

Clause 19: The method of any one of Clauses 5-18, further comprising generating training data based on the prior user data, comprising: identifying a set of records, in the prior user data, indicating that a corresponding user has depression, based on searching the prior user data for a defined medical code; and for each respective record in the set of records, extracting a respective set of user attributes corresponding to a defined window of time prior to a time associated with the respective record.

Clause 20: A system, comprising: a memory comprising computer-executable instructions; and one or more processors configured to execute the computer-executable instructions and cause the processing system to perform a method in accordance with any one of Clauses 1-19.

Clause 21: A system, comprising means for performing a method in accordance with any one of Clauses 1-19.

Clause 22: A non-transitory computer-readable medium comprising computer-executable instructions that, when executed by one or more processors of a processing system, cause the processing system to perform a method in accordance with any one of Clauses 1-19.

Clause 23: A computer program product embodied on a computer-readable storage medium comprising code for performing a method in accordance with any one of Clauses 1-19.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system 135) or related data available in the cloud. For example, the machine learning system 135 could execute on a computing system in the cloud and train and/or use machine learning models. In such a case, the machine learning system 135 could train models to generate depression risk scores, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
receiving user data describing a first user;
extracting, from the user data, a first set of user attributes corresponding to a defined set of features, wherein at least a first attribute of the first set of user attributes is generated by processing unstructured text using a first trained machine learning model;
training a second machine learning model to generate risk scores based on the first set of user attributes, wherein the risk scores indicate probability that users have or will develop depression; and
deploying the second trained machine learning model.

2. The method of claim 1, wherein extracting the first set of user attributes comprises:
identifying a plurality of notes in the user data, wherein each of the plurality of notes comprises natural language text describing the first user;
generating a set of scores for the plurality of notes by processing the plurality of notes using the first trained machine learning model; and
aggregating the set of scores to generate the first attribute.

3. The method of claim 2, wherein generating the set of scores by processing the plurality of notes comprises preprocessing at least a first note of the plurality of notes, comprising:
normalizing natural language text in the first note; and
converting the normalized natural language text in the first note to a numerical vector.

4. The method of claim 1, wherein training the second machine learning model further comprises:
generating training data based on prior user data, the prior user data indicating a respective set of user attributes for each respective user of a plurality of users, comprising:
identifying a set of records, in the prior user data, indicating that a corresponding user has depression, based on searching the prior user data for a defined medical code; and
for each respective record in the set of records, extracting a respective set of user attributes corresponding to a defined window of time prior to a time associated with the respective record.

5. A method, comprising:
receiving user data describing a first user;
extracting, from the user data, a first set of user attributes corresponding to a defined set of features, wherein at least a first attribute of the first set of user attributes is generated by processing unstructured text using a first trained machine learning model;
generating a first risk score by processing the first set of user attributes using a second trained machine learning model, wherein the first risk score indicates a probability that the first user has or will develop depression; and
initiating one or more interventions for the first user based on the first risk score.

6. The method of claim 5, wherein extracting the first set of user attributes comprises:
identifying a plurality of notes in the user data, wherein each of the plurality of notes comprises natural language text describing the user;
generating a set of scores for the plurality of notes by processing the plurality of notes using the first trained machine learning model; and
aggregating the set of scores to generate the first attribute.

7. The method of claim 6, wherein generating the set of scores by processing the plurality of notes comprises preprocessing at least a first note of the plurality of notes, comprising:
normalizing natural language text in the first note, and
converting the normalized natural language text in the first note to a numerical vector.

8. The method of claim 5, further comprising:
determining that the first risk score exceeds a defined threshold; and
generating an alert identifying the first user.

9. The method of claim 8, further comprising:
identifying a most impactful attribute, from the first set of user attributes, that caused the first risk score to exceed the defined threshold; and
indicating the most impactful attribute in the generated alert.

10. The method of claim 5, further comprising:
for each respective user of a plurality of users in a healthcare facility:
identifying a respective set of user attributes; and
generating a respective risk score for the respective user by processing the respective set of user attributes using the second trained machine learning model.

11. The method of claim 5, wherein the defined set of features comprises:
one or more features relating to diagnoses,
one or more features relating to clinical assessments, and
one or more features relating to medications.

12. The method of claim 11, wherein:
the one or more features relating to diagnoses comprise a defined set of diagnoses, and
the first set of user attributes indicates, for each respective diagnosis of the defined set of diagnoses, whether the first user has the respective diagnosis.

13. The method of claim 12, wherein the first set of user attributes further indicates, for each respective diagnosis of the defined set of diagnoses, whether the first user was diagnosed with the respective diagnosis within a defined window of time.

14. The method of claim 11, wherein:
the one or more features relating to clinical assessments comprise a defined set of conditions, recorded by one or more caregivers, relating to functional states of users, and
the first set of user attributes indicates, for each respective condition of the defined set of conditions, whether the first user has the respective condition.

15. The method of claim 14, wherein the defined set of conditions comprises at least one of: (i) weight loss, (ii) weight gain, (iii) pain, (iv) increased food intake, (v) decreased food intake, (vi) isolation, or (vii) one or more mood or behavioral issues.

16. The method of claim 11, wherein:
the one or more features relating to medications comprise a defined set of medications, and
the first set of user attributes indicates, for each respective medication of the defined set of medications, whether the first user receives the respective medication.

17. The method of claim 16, wherein the first set of user attributes further indicates, for each respective medication of the defined set of medications, whether the first user was prescribed the respective medication within a defined window of time.

18. The method of claim 5, wherein the first second machine learning model was trained on prior user data for a plurality of users, the prior user data indicating a respective set of user attributes for each respective user of the plurality of users.

19. The method of claim 18, wherein the training data was generated based on the prior user data, by:
identifying a set of records, in the prior user data, indicating that a corresponding user has depression, based on searching the prior user data for a defined medical code; and
for each respective record in the set of records, extracting a respective set of user attributes corresponding to a defined window of time prior to a time associated with the respective record.

20. A non-transitory computer-readable storage medium comprising computer-readable program code that, when executed using one or more computer processors, performs an operation comprising:
receiving user data describing a first user;
extracting, from the user data, a first set of user attributes corresponding to a defined set of features, wherein at least a first attribute of the first set of user attributes is generated by processing unstructured text using a first trained machine learning model;
generating a first risk score by processing the first set of user attributes using a second trained machine learning model, wherein the first risk score indicates a probability that the first user has or will develop depression; and
initiating one or more interventions for the first user based on the first risk score.
